(19) United States
(12) Patent Application Publication
(10) Pub. No.: US 2018/0263483 A1
(43) Pub. Date: Sep. 20, 2018

(54) Dental Mirror Device with Affixed Camera

(71) Applicant: Dental SmartMirror, Inc., Chicago, IL (US)

(72) Inventor: Gidon Oded Elazar, Cohav Yair (IL)

(21) Appl. No.: 15/985,052

(22) Filed: May 21, 2018

Related U.S. Application Data
(63) Continuation-in-part of application No. 15/360,479, filed on Nov. 23, 2016.
(60) Provisional application No. 62/354,694, filed on Jun. 25, 2016.
(60) Provisional application No. 62/354,273, filed on Jun. 24, 2016.
(60) Provisional application No. 62/343,034, filed on May 30, 2016.
(60) Provisional application No. 62/341,629, filed on May 26, 2016.

(51) Int. Cl. A61B 1/247; A61B 1/05; A61B 1/06

(52) U.S. Cl. CPC A61B 1/247; H05B 37/0227; A61B 1/0676; A61B 1/05

(57) ABSTRACT

In an embodiment, a dental mirror includes an image sensor, a plurality of light sources, a mirror surface, an appendage affixed to the mirror surface to extend the mirror surface into a patient's mouth, and a handle affixed to the appendage. The mirror surface includes a transparent planar material covering the image sensor and the plurality of light sources, and a reflective coating portion on the transparent planar material. The plurality of light sources are positioned around a perimeter of the mirror surface close enough to one another to emit a continuous ring of light.

This application is a continuation-in-part of U.S. application Ser. No. 15/360,479, filed Nov. 23, 2016, which claims benefit of U.S. App. No. 62/354,694, filed Jun. 25, 2016, U.S. App. No. 62/354,273, filed Jun. 24, 2016, U.S. App. No. 62/343,034, filed May 30, 2016, and U.S. App. No. 62/341,629, filed May 26, 2016, all of which are incorporated by reference in their entirety.

### BACKGROUND

### Field

This field is generally related to dental instruments.

### Related Art

Intraoral mirrors, also known as mouth mirrors, are among the most functional and frequently used of dental instruments. Viewing objects in a mouth directly is difficult due to a limited, or perhaps nonexistent, line of sight. Intraoral mirrors allow a health care provider (HCP), for example dentist, hygienist and others, to indirectly view teeth and other objects in a patient's mouth, such as the patient's gums and tongue, by observing their reflections in a mirror. Health care providers use the intraoral mirror for a variety of tasks, including, but not limited to, evaluation and diagnosis, treatment selection, and even to assist the treatment itself. A health care provider may use other tools, such as a dental hand piece, in conjunction with the mirror to conduct procedures, such as tooth preparation, when the procedures are conducted in areas that are not directly visible.

Not only are they used as a visual aid, intraoral mirrors are also used as rigid tools to manipulate or protect objects in a patient's mouth. For example, a health care provider may use an intraoral mirror to shift a patient's cheek to make space for treatment or to expand the mouth space for improved visibility. In addition, an intraoral mirror can protect soft and hard tissue structures of a patient's mouth while other parts of the mouth are treated.

Since an intraoral mirror is in contact with a patient's tissues inside their mouth, the mirror goes through sterilization after each treatment. In some cases, sterilization is done using a process known as “autoclaving.” Autoclaving subjects the mirror to high temperature and pressure, perhaps using steam. Because the mirror must be sterilized after each treatment, a dental office possesses multiple such mirrors.

The mirror, made mostly of glass and metal, can withstand the autoclaving process. To make the glass reflective, it is often coated with chromium, which is chosen because it can withstand autoclaving. But, due to frequent use and sterilization, the mirror eventually loses some of its clarity and its reflectiveness, thus needing replacement. A common hurdle while using mirrors is that they tend to fog up as they heat in the warm, humid environment of the patient's mouth.

Typically, a dentist illuminates the patient's mouth using overhead lights. However these are cumbersome and inaccurate. While dental mirrors with LED lights may also be available, LEDs can take up valuable space on the mirror that would otherwise be occupied by a reflective coating.

In addition to intraoral mirrors, intraoral cameras are becoming more widespread in dental clinics. Intraoral cameras have principally two uses. First, intraoral cameras are used to describe a diagnosis and explain a possible treatment to a patient. For example, to explain a diagnosis or treatment, the health care provider may show images of the patient's mouth parts (e.g. teeth) to the patient. Second, the intraoral cameras are used to record the state of portions of the patient's mouth. For example, a health care provider may capture a photographic image of the patient's mouth before or after treatment.

Intraoral cameras are commonly shaped as pens, with an image capture device at their tip, pointed sideways. The tip helps orient the HCP as to where to position the camera to capture images of the desired area of the mouth. The captured images are presented in a display, and the navigation is done using the display. However, because these displays are the only viewfinder for the cameras, their use adds time to a dental appointment. Additionally, in a common usage scenario, heath care providers would commence a mouth inspection using a mouth mirror, if a need to capture an image arises, the health care provider would have to switch the intraoral mirror with an intraoral camera. This may seem a minor hassle, but in the busy environment of a dental clinic, it reduces the frequency of capturing images.

Some dental procedures use dental composite resin material to glue fillings or build up structures on teeth during restoration procedures. After applying the material it is hardened using an instrument called a light cure. The light cure is used to illuminate the resin with light within the spectrum of visible blue to ultraviolet. This light might be harmful to the eye, therefore an eye protector is used by the healthcare provider while using the light cure. To perform such procedure, a health care provider applies the resin to the teeth. In many cases, to observe the tooth of interest, a health care provider uses a mouth mirror while applying the resin. When done, the health care provider switches instrument to a light cure, and illuminates the area for the resin to cure. When building up material on a tooth, the process repeats so that resin is applied, followed by curing, and then applied again, requiring to repeatedly switch the mouth mirror and light cure instruments.

Systems and methods are needed to provide a more efficient way to show a patient images from the patient's mouth.

### BRIEF SUMMARY

Embodiments integrate a camera into an intraoral mirror. Integrating a camera into an intraoral mirror provides an efficient way to record and display what is visible to the healthcare provider in the mirror.

In an embodiment, a dental mirror includes an image sensor, a plurality of light sources, a mirror surface, an appendage affixed to the mirror surface to extend the mirror surface into a patient's mouth, and a handle affixed to the appendage. The mirror surface includes a transparent planar material covering the image sensor and the plurality of light sources, and a reflective coating portion on the transparent planar material. The plurality of light sources is positioned around a perimeter of the mirror surface close enough to one another to emit a continuous ring of light.

Other device, system, method, and computer program product embodiments are also disclosed.

Further embodiments, features, and advantages of the invention, as well as the structure and operation of the various embodiments, are described in detail below with reference to accompanying drawings.

### BRIEF DESCRIPTION OF THE DRAWINGS

The accompanying drawings, which are incorporated herein and form part of the specification, illustrate the present disclosure and, together with the description, further serve to explain the principles of the disclosure and to enable a person skilled in the relevant art to make and use the disclosure.

FIG. 1 is a diagram illustrating components of an intraoral mirror including an integrated camera, according to an embodiment.

FIG. 2A is a diagram illustrating a cross-section of a mirror portion of the intraoral mirror.

FIGS. 2B-C are diagrams illustrating a mirror surface, according to embodiments.

FIG. 3 is diagram illustrating an intraoral mirror being used to examine a patient's mouth from the perspective of a healthcare provider.

FIG. 4 is a diagram illustrating how light from the patient's mouth is reflected off the mirror's reflective surface and observed by a healthcare provider.

FIG. 5 is a diagram illustrating a base station, according to an embodiment.

FIG. 6 is a diagram illustrating a cross-section of an intraoral mirror illustrating how to avoid sound reverberation, according to an embodiment.

FIG. 7A is a diagram illustrating a disposable sleeve for an intraoral camera, according to an embodiment.

FIG. 7B is a diagram illustrating an intraoral camera with a disposable sleeve attached, according to an embodiment.

FIG. 8A illustrates a room of a dentist office including a system for displaying what is apparent to the healthcare provider in an intraoral mirror.

FIG. 8B is an architecture diagram of the system in FIG. 8A.

FIG. 9 is a block diagram of the intraoral mirror including an integrated camera.

FIG. 10 illustrates a method for monitoring usage of the intraoral mirror.

The drawing in which an element first appears is typically indicated by the leftmost digit or digits in the corresponding reference number. In the drawings, like reference numbers may indicate identical or functionally similar elements.

### DETAILED DESCRIPTION

Embodiments are described that overcome some of the disadvantages described above. For example, a dental mirror may include LED lights that are partially covered with a reflective material. This is achieved by creating a lattice form in the reflective material in the corresponding area of the mirror surface. The lattice might be a checkers form, where blacks have reflective material and whites do not.

In an embodiment, sapphire instead of glass is used as a base for the mirror reflector. Sapphire is very hard, harder than glass, enabling it to withstand the tough environment of use at the dental office and even the touch of tools that work in proximity. Sapphire also allows the mirror to be much thinner. Having thickness smaller than 1 mm allows the sapphire to be back-coated with the reflective material. Coating on the back or interior side of the transparent surface may be preferable for use of the LEDs and for integration of an image sensor. Placing the reflective coating on the interior side also protects the coating itself from scratches.

In an embodiment, the mirror may be accompanied with a disposable, single use sleeve adapted to cover the portion of the mirror that enters a patient's mouth. By using a disposable sleeve, embodiments avoid the need for autoclaving.

In an embodiment, the transparent, sapphire surface may be coated with aluminum to make it reflective. Aluminum has a higher reflectivity than the typical chromium, making for a clearer image. Aluminum may not withstand autoclaving as well as chromium. However, the ability to withstand autoclaving is no longer required when a disposable sleeve is used.

These and other aspects are discussed with respect to individual embodiments below.

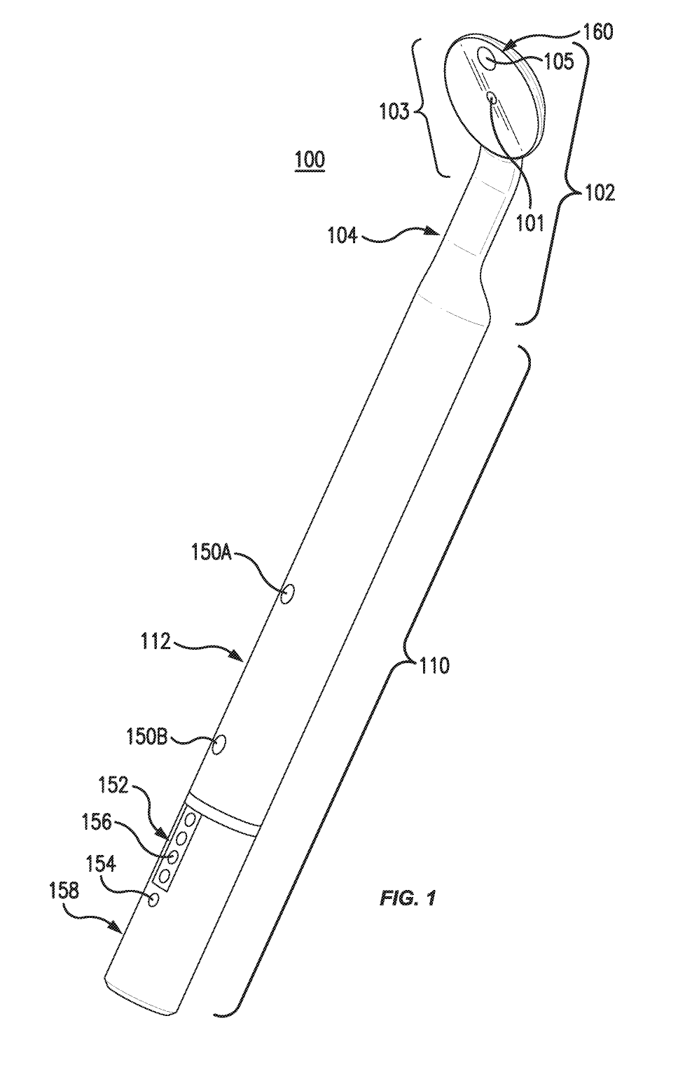

FIG. 1 is a diagram illustrating components of an intraoral mirror including an integrated camera, referred to herein as a smart mirror device, according to an embodiment. In particular, FIG. 1 illustrates physical components of a smart mirror device 100.

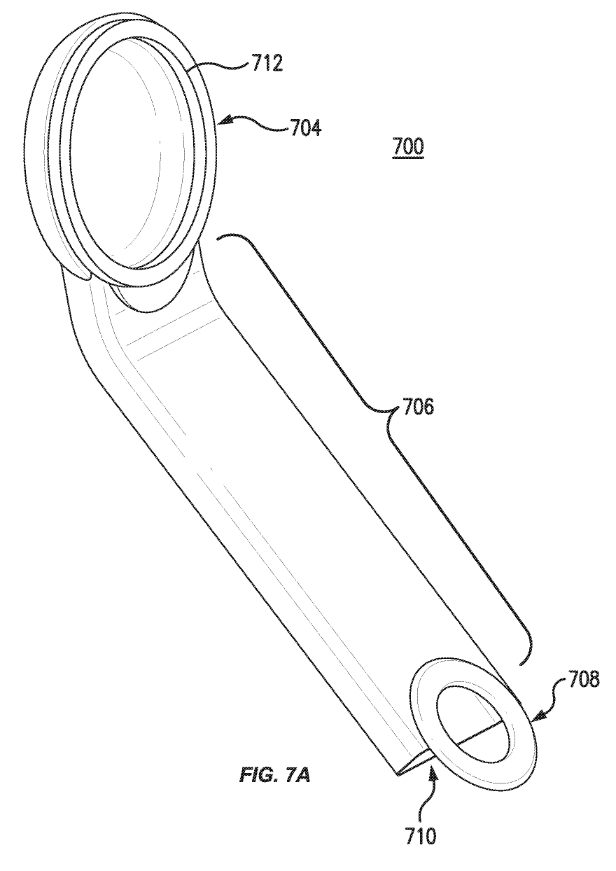

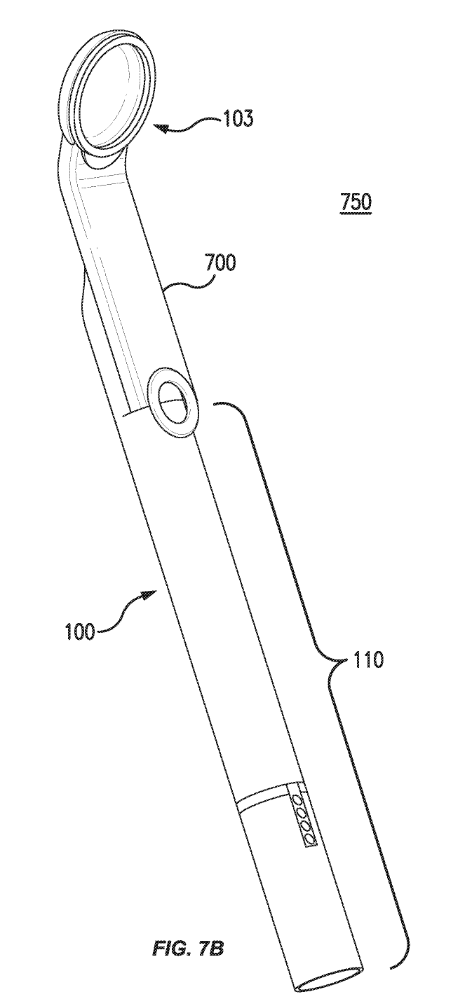

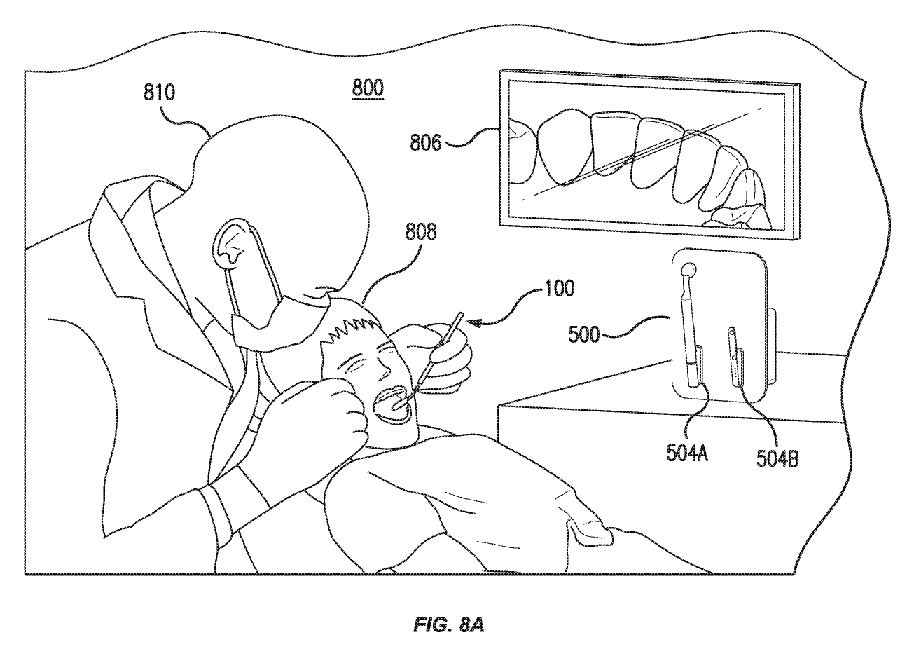

Smart mirror device 100 includes an oral piece 102 and a hand piece 110. Oral piece 102 may be the portion of smart mirror device 100 that is designed to enter a patient's mouth. As described below with respect to FIGS. 7A and 7B, oral piece 102 may be covered with a single use disposable sleeve to maintain sterility. By using a sterile, disposable sleeve, embodiments avoid the need for smart mirror device 100 to the autoclaved.

In contrast to oral piece 102, hand piece 110 is not adapted to be in contact with a patient's mouth. Because hand piece 110 is not in contact with a patient's mouth, it may not need to be as sterile as oral piece 102, and hence may not need to be covered with a sleeve.

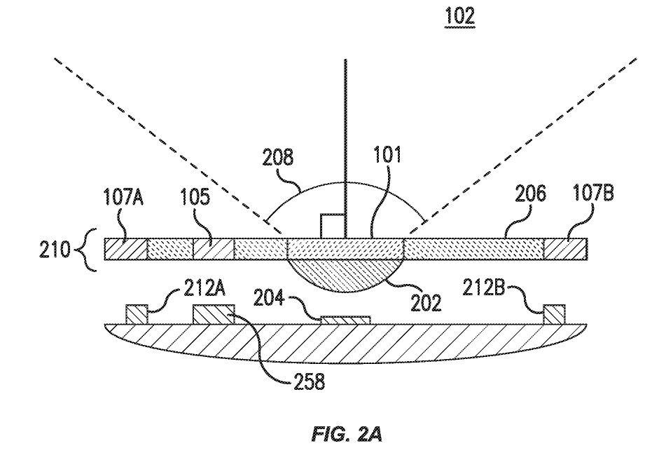

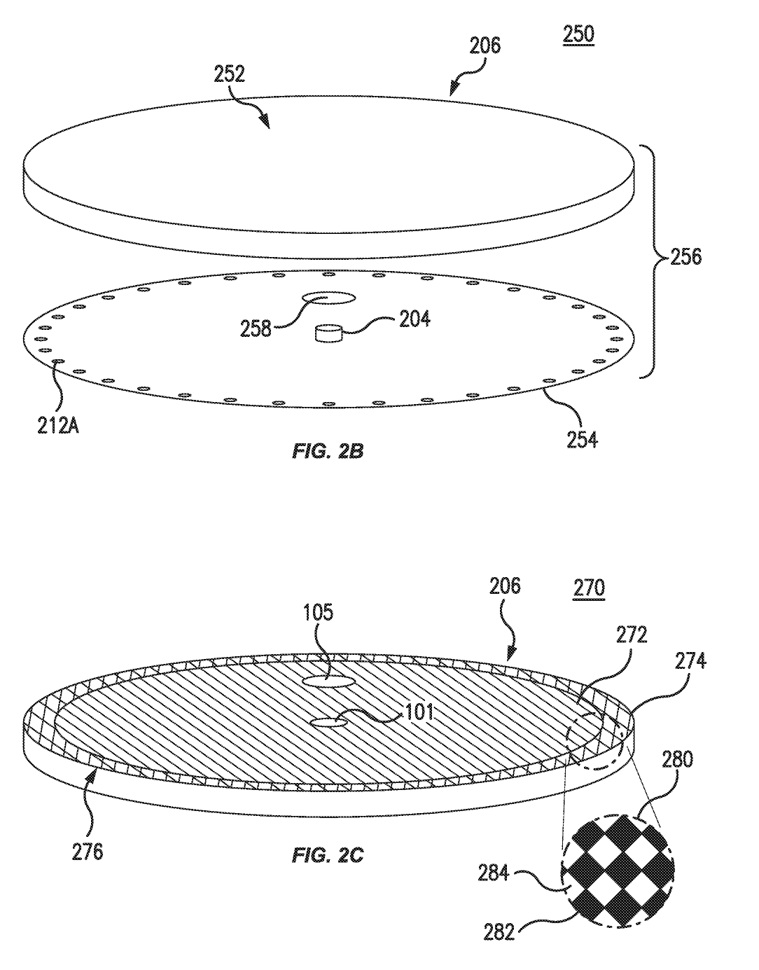

Returning to oral piece 102, oral piece 102 includes a viewfinder mirror 103 and an appendage 104. Viewfinder mirror 103 is a mirror, and hence has a reflective surface 160. In some embodiments, viewfinder mirror 103 could include electronic components. Viewfinder mirror 103 may be round in shape. Viewfinder mirror 103's reflective surface includes pass-through surfaces 101 and 105. Pass-through surfaces 101 and 105 may be circular. They may be transparent or semi-reflective, allowing light to reach an image sensor (not shown) in oral piece 102 or allowing light to pass-through from a curing light source. In one embodiment, each of pass-through surfaces 101 and 105 may be a clear transparent window small enough to enable a healthcare provider to have a clear view of the reflection when observing the reflective surface. A semi-reflective surface appears reflective from the outside of the smart mirror device 100. But, from behind a semi-reflective surface, the surface is transparent. The lattice described below with respect to FIG. 2C is an example of a semi-reflective surface.

In an embodiment, viewfinder mirror 103 is round and pass-through 101 is located substantially at the center of mirror so that it appears at the center to the naked eye. Viewfinder mirror image 103 provides visual guidance to a user about the objects that may be included in images captured by the sensor concealed behind pass-through surface 101. Hence, when smart mirror device 100 is being used by a health care provider in an intraoral environment, the image sensor (not shown) behind pass-through surface 101 may capture images that include at least some of the objects whose reflection can be observed in the viewfinder mirror 103.

In an embodiment, pass-through surface 105 is located off-center and may allow light to emit, from a light source (not shown) in oral piece 102 to cure a dental composite resin. When this light source is activated, light is emitted through pass-through surface 105 at a frequency adapted to cure dental resin.

Appendage 104 is affixed to viewfinder mirror 103, protecting hand piece 110 from contacting the intraoral environment while allowing for comfortable intraoral use of viewfinder mirror 103. Appendage 104 has an elongated shape with viewfinder mirror 103 affixed to one end of the shape, extending viewfinder mirror 103 into the patient's mouth. In some embodiments, appendage 104 has a shape of a tube or cylinder, but other shapes, including those with non-rounded cross-sections, are possible. In some embodiments, at least part of appendage 104 is hollow, allowing space for electronic components. In some embodiments, electronic components are located elsewhere, the invention is not so limited.

While one end of appendage 104 is affixed to viewfinder mirror 103, the opposite end is affixed to hand piece 110. Hand piece 110 includes handle 112 and end piece 158. Handle 112 enables a health care provider to grasp smart mirror device 100. Like appendage 104, handle 112 may have an elongated shape, such as a cylinder, and may be hollow on the inside to conceal electronic components. For example, handle 112 may conceal a power supply, a processor, and accelerometer and/or gyroscope and/or magnetometer. In other embodiments, these components may be located in oral piece 102. In an embodiment, handle 112 may include a shape substantially like an elongated oval, such as an oval cylinder, which may allow a more comfortable grasp than a circular cylinder.

Handle 112 includes a plurality (in this case, two) of magnets 150A-B. Magnets 150A-B may be concealed inside handle 112. Magnets 150A-B serve to attach smart mirror device 100 to a base station. Magnets 150A-B may be positioned such that, when placed on the base station, smart mirror device 100 connects properly. For example, magnets 150 A-B may force a connector 152 on smart mirror device 100 into the correct location, while compensating for user placement error. Magnets 150A-B also provide sufficient force to press connector 152 to corresponding pins on the base station, ensuring there is electronic connectivity. Magnets 150A-B also allow a health care provider to choose the placement of the base station, with the plane substantially parallel or vertical to the table. They allow the health care provider to position the base station in a way where smart mirror device would fall from force of gravity, were the magnets 150A-B not holding it in place. At the same time, magnets 150A-B may be sufficiently weak to allow a health care practitioner to pick up the mirror off the base station effortlessly.

Handle 112 and appendage 104 may be made of metal, such as a surgical grade stainless steel. For example, they may be made of austenitic 316 stainless steel, or martensitic 440 or 420 stainless steels. The metal also dissipates heat generated by the electronic components. Generally, an undesirable side effect of producing heat is present during the operation of electronic components, for example the camera sensor or the accelerometer. Where handle 112 or appendage 105 made of a material other than metal that did not conduct heat and allow it to dissipate well, heat could potentially accumulate inside the oral piece 102 or hand piece 110. The accumulated heat would gradually raise the temperature of the dental mirror, becoming uncomfortable to touch a patient mouth or the heath care provider's hand. To reduce the temperature the heat may be conducted to flow to the handle, and dissipate to the air. Similarly, the design could enable heat generated by the electronics in the handle to flow to the area around the mirror in order to raise its temperature, if desired. The heat flow may be controlled, for example, by the geometric shape and amount of steel present. It may be controlled in such a way that the temperature of the Sapphire remains around 26-32 degrees Celsius. This temperature range, which is below the human body temperature, has the much desirable effect of avoiding the build up of fog on the mirror's surface while still being comfortable for the patient mouth to touch.

However, smart mirror device 100 may include electronic components that have performance problems from enclosure in metal. For example, smart mirror device 100 may include a wireless transmitter (not shown). For a wireless transmitter, enclosure in a conductive material, such as steel, may cause interference. The conductive material may act as a Faraday cage that blocks or disrupts some transmissions.

Thus, smart mirror device 100 may include an end piece 158 that is made of the nonconductive material, such as plastic or perhaps ceramic. End piece 158 may include at least those electronic components, such as a wireless transmitter, that may suffer from performance issues from enclosure in metal. In FIG. 1, end piece 158 also includes connector 152 and a hole 154.

Connector 152 includes pins 156 that couple to a base station (not shown) and allow transmission of power, and perhaps data, from the base station to smart mirror device 100. Pins 156 may provide a USB connection. For example, connector 152 may include four pins 156, one for voltage (e.g., +5 V), one for ground, and two for data.

Hole 154 may provide an entry point for sound to enter end piece 158. End piece 158 may include a microphone, and hole 154 may allow sound waves to enter end piece 158 so that they can be detected by the microphone. Hole 154 can be somewhere else in smartmirror 100. It is preferable to place it on end piece 158, because (1) it avoids the need to pierce the metal, thus keeping it better sealed, (2) it is in an area that is not covered by a disposable sleeve, and (3) the doctor's hand does not cover the hole while grasping the mirror.

FIG. 2A is a diagram illustrating a cross-section of at least a portion of oral piece 102 of the smart mirror device 100 shown in FIG. 1. Oral piece 102 includes a mirror surface 206 and may include a transparent planar material 210 that is planar in shape. As described in more detail below, transparent planar material 210 may be made of a transparent material, such as synthetic sapphire, that is coated with a reflective material. Sapphire is very hard, harder than glass, enabling mirror surface 206 to withstand the tough environment and use at the dental office and even the touch of tools that work in proximity. Sapphire also enables transparent planar material 210 to be very thin, for example less than 1 mm in thickness. In an embodiment, transparent planar material 210 may be approximately 0.3 mm. The thinness of transparent planar material 210 allows for a back surface mirror. A back surface mirror introduces a secondary image reflection since some of the light is reflected by the transparent front of the mirror, before it reaches the reflective surface at the back. However, such a thin planar material reduces this effect to become negligible. As mentioned above, coating on the back or interior side of the transparent surface may be preferable for use of the LEDs and for integration of an image sensor. Placing the reflective coating on the interior side also protects the coating itself from scratches.

As will be described below, in addition to transparent planar material 210, mirror surface 206 includes a reflective coating that reflects light into a health care provider's field of view.

As mentioned above for FIG. 1, mirror surface 206 comprises pass-through surface 101, such as an opening in the reflective coating, that is configured such that at least some of the light that reaches the opening actually arrives at an image sensor. In other words, one side is transparent to image sensor 204.

Around a perimeter of mirror surface 206 may be a plurality of light sources 212A-B. Light sources 212 may be illumination devices, such as light emitting diodes. Light sources 212 may each be situated behind a partially reflective surface 107A-B. The partially reflective surface 107A-B may appear reflective to a healthcare provider looking at viewfinder mirror 103. However, they allow light from light sources 212 to be transmitted through mirror surface 206. In this way, light sources 212A-B illuminate the interior of the patient's mouth, while being concealed and while preserving space on mirror surface 206.

The light sources 212 might be affixed around and beneath the perimeter of viewfinder mirror 103. Light source 212 illuminates the intraoral environment. In some embodiments, light source 212 illuminates in the visible light spectrum, for example a light of similar color hues of daylight, which enables a visual perception (as well as capturing of images) of natural colors, or maybe a so-called “warm white” color which in some cases produces an illumination which is more comfortable to the human eye, or perhaps a “cooler” white to which tends to produce a light environment that is considered “crisp”. In some embodiments, light source 212 illuminates in non-visible radiation, for example in a frequency within the infrared spectrum.

Viewfinder mirror 103 may also include a light source 258. Light source 258 may be an illumination device, such as light emitting diodes, situated behind pass-through 105. Light source 258 illuminates the intraoral environment with a light frequency configured to cure dental resin. The light used may fall under the visible blue light spectrum. In particular, light source 258 may produce a narrow spectrum of blue light in the 400 nm to 500 nm range (with a peak wavelength of about 460 nm), which is the useful energy range for activating molecules commonly used to initiate the photo-polymerization of dental monomers. Light source 258 may, for example, use a gallium nitride-based semiconductor for blue light emission. In a further embodiment, a red light may be used for illuminating during curing placement.

Situated between image sensor 204 and pass-through 101 is a lens 202. Lens 202 may refract light to image sensor 204 from a field of view 208. Lens 202's field of view 208 has a viewing angle which may be wide enough to simultaneously capture both part of a face of a health care provider and a portion of a patient's mouth that is visible to the health care provider in some intraoral usage conditions. If the face of a health care provider is not visible to image sensor 204, the last known position of the health care provider's face or eyes is used.

In various embodiments, the viewfinder mirror 103 may have several lenses. The multiple lenses may move or adjust to allow focus at different distances. There may be a mechanism for autofocus or a mechanism to add or remove one or more lenses between the mirror surface and the sensor.

In additional embodiments, light from pass-through 101 may be split to multiple structures of lenses and sensors. In addition, the light from pass-through 101 may be folded before the light reaches an image sensor 204 using a prism. In this way, the image sensor 204 may be located in an appendage or handle and still capture light through pass-through 101.

FIGS. 2B-C are diagrams illustrating a viewfinder mirror according to embodiments. FIG. 2B includes a diagram 250 illustrating an assembly 256 to be used in viewfinder mirror 103. Assembly 256 includes mirror surface 206 and a backing 254. Mirror surface 206 includes an exterior side 252 visible from the perspective shown in diagram 250. Exterior side 252 may be the portion of mirror surface 206 exposed to the outside of viewfinder mirror 103. As described above, mirror surface 206 may be made of a transparent planar material, such as a transparent synthetic sapphire. In an embodiment, no reflective coating is applied to exterior side 252. As mentioned above, mirror surface 206 may be circular. It may be between 18 mm and 24 mm in diameter, to accommodate to various human oral cavity dimensions, and is preferably about 22 mm in diameter.

Backing 254 may, for example, be a printed circuit board (PCB) that mechanically supports and electrically connects various electronic components of smart mirror device 100, including light sources 212 and 258, and image sensor 204. In FIG. 2B, image sensor 204 is positioned substantially at the center of backing 254, and light sources 212 are positioned around the perimeter of backing 254, distributed substantially evenly around the perimeter. In an embodiment, backing 254 may include between 20 and 70 LED lights, preferably between 20 and 40. The LED lights may be positioned to emit a continuous ring of light. To an observer, the ring may not appear as individual lights, but as a continuous ring. To create the continuous ring, the light sources on backing 245 may be spaced no more than 18 degrees apart. They may be spaced approximately evenly, with at least 20 LED lights. In a preferred embodiment, backing 254 may include 36 LED lights, approximately evenly spaced every 10° around the 360° circular mirror. In another embodiment, the LEDs may for an arc smaller than 360 degrees around the perimeter. In that embodiment, the arc does not close to a complete ring. For example, the LED lights may form a 270 degrees arc, where the 90 degree portion closer to the handle has no LEDS.

FIG. 2C illustrates a diagram 270 that illustrates an interior side 276 of mirror surface 206. Interior side 276 is on the reverse side relative to exterior side 252. Interior side 276 may be the portion of mirror surface 206 that is enclosed on the interior of viewfinder mirror 103. Interior side 276 includes reflective coatings applied to the transparent planar material. In particular, interior side 276 may include two different portions with different reflective coatings: coating portion 272 and coating portion 274.

Reflective coating portion 272 is positioned to reflect light into a health care provider's field of view. Reflective coating portion 272 may include pass-through 101 positioned to allow the image sensor to capture an image of what is reflected into the health care provider's field of view and (optionally) pass-through 105 positioned to allow transmit light to cure dental resin. As a gap in the coating, pass-throughs 101 and 105 may act as windows. Pass-throughs 101 and 105 may be circular portions of interior side 276.

Pass-through surface 101 may be sized according to the size of lens 202 between mirror surface 206 and image sensor 204 in viewfinder mirror 103 illustrated in FIG. 2A. Pass-through surface 101 may be as small as possible such that it still allows light from field of view 208 to be collected by lens 202 and refracted toward image sensor 204. In this way, pass-through 101 may be only a mere pinhole, maximizing the area available for reflective coating portion 272. Pass-through 101 may be between 0.8 mm and 1.3 mm in diameter, with the diameter preferably around 1.2 mm.

Reflective coating portion 272 may be a solid, continuous coating of aluminum. Aluminum has a high reflectivity higher than the typical chromium, making for a clearer image. Aluminum may not withstand autoclaving as well as chromium. However, the ability to withstand autoclaving is not required when a disposable sleeve is used, as discussed above and below.

Reflective coating portion 274 is positioned around the perimeter of mirror surface 206 to cover the light sources 212 shown in FIG. 2B. Reflective coating portion 274 may also be of aluminum but may differ from reflective coating portion 272 in that it may not be a solid coating, and instead being applied non-continuously with discontinuities allowing some light to pass through. Reflective coating portion 274 may be positioned to allow light sources 212 to emit light through the reflective coating portion 274 to illuminate what is reflected into the field of view. Reflective coating portion 274 may have a shape of a ring around the perimeter, to allow an adequate light distribution. The ring's width may be between 0.4 mm and 1.5 mm in width, with the width preferably around 0.5 mm. The width may be selected to optimize the amount of light allowed to pass and to optimize its spread angle without much reduction to the in the reflection area.

Not being solid, reflective coating portion 274 may be applied in a pattern, for example a lattice pattern with some boxes in the lattice being coated with aluminum and others being left clear. Such a checkerboard pattern is illustrated in close up 280. Close up 280 shows an enlarged view of the lattice, checkerboard pattern with alternating boxes of that are either left clear (as in box 282) or coated with aluminum (as in box 284). But boxes coated with aluminum need not touch each other's corners. As the boxes coated with aluminum are further from each other, reflective coating portion 274 becomes less reflective and allows more light from lighting sources 212 to transit. As a corollary, as the boxes coated with aluminum are closer to each other, reflective coating portion 274 becomes more reflective and allows less light from lighting sources 212 to transit.

Because the lattice is too small to be seen by the naked eye, when light source 212 is not illuminated, reflective coating portion 274 reflects light into the health care provider's field of view, appearing to the healthcare provider as simply a mirror, with possibly reduced reflection when compared to 272, but concealing the presence of the light sources behind it. However, when light source 212 is illuminated, reflective coating portion 274 allows light to emit from the light source 212 to illuminate the interior of a patient's mouth, with some reduction to the intensity of the illumination when compare to a fully clear ring.

Again, reflective coating portion 274 is optional. In another embodiment, the ring may be fully clear.

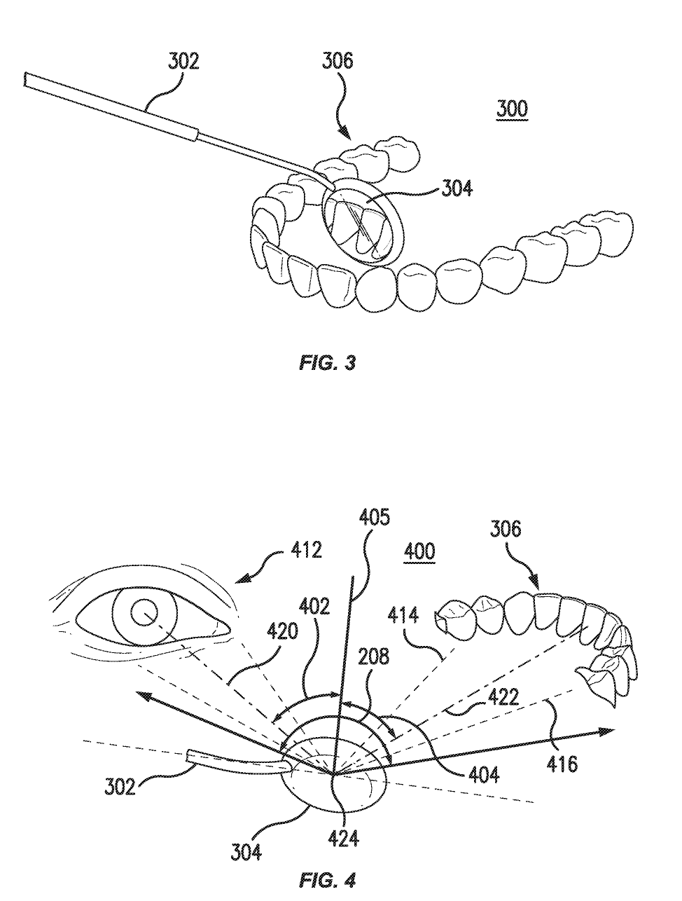

FIG. 3 shows diagram 300 illustrating an intraoral mirror being used to examine a patient's mouth from the perspective of a healthcare provider. In particular, diagram 300 includes smart mirror device 302 having a mirror surface 304. In diagram 300, smart mirror device 302 is being used to inspect teeth 306. In diagram 300, mirror surface 304 is positioned to show the lingual surface of teeth 306 (the back of the tooth) from the healthcare provider's perspective. Notably, what is visible from the healthcare provider's perspective may differ from what is visible from the image sensor's perspective. This is illustrated, for example, in FIG. 4.

FIG. 4 shows a diagram 400 illustrating how light from a patient's mouth is reflected off a mirror's reflective surface and observed by a healthcare provider. Diagram 400 shows how smart mirror device 302's mirror surface 304 reflects light from the lingual surface of teeth 306 to an eye of a healthcare provider 412. In particular, a plurality of rays of light spanning from ray 414 to ray 416 travel from teeth 306 to mirror surface 304. Each of the rays, which we shall call incident rays, reach mirror surface 304 at an incidence angle, for example ray 422 at incidence angle 404. Then, according to the so-called “law of reflection”, the rays are reflected from the mirror surface 304 so that each reflected ray, the respective incident ray and the normal to the surface 405 at the incidence point (labeled incidence point 424) are all on the same plane. Moreover, the angle of reflection is equal to the angle of incidence. The combination of reflected rays that happen to reflect toward the healthcare provider 412 define what is being observed in the mirror. For example, incident ray 422 reaches the mirror surface 304 at an incidence point on mirror surface 304 and at an incidence angle 404 and its reflected ray 420 towards the healthcare provider 412 is at a reflection angle 402 which is equal to the incidence angle 404. Thus, the lingual surface of teeth 306 is visible to healthcare provider 412, and health care provider 412 observes the perspective illustrated in FIG. 3.

As described above for FIG. 2, smart mirror device 302 includes an image sensor, and the image sensor captures light refracted by a lens with field of view 208. As illustrated in diagram 400, field of view 208 is wide enough to capture the lingual surface of teeth 306, which is visible to healthcare provider 412 on mirror surface 304, even in cases where healthcare provider 412 is focusing on incidence points off the center of the mirror. Thus a health care provider may use a smart mirror device 302 to capture image snapshots without interrupting treatment by switching to a separate intraoral camera instrument. Nonetheless, the captured image must be processed so that the result fits what healthcare provider 412 observes in the mirror surface 304. This processing is required because the orientation of an intraoral mirror may change as a healthcare provider rotates it and also because possibly more is visible to the image sensor in smart mirror device 302 than is visible to healthcare provider 412. For example, field of view 208 is wide enough to capture part of the face of health care provider 412, while health care provider 412 may not witness his reflection in mirror surface 304. Therefore, in order to improve the use of the intraoral mirror as a viewfinder, the portion of an image captured by the image sensor that is being observed by the healthcare provider 412 must be determined.

To determine which portion of field of view 208 is being observed by healthcare provider 412, healthcare provider 412's face may be detected in the image captured by the image sensor. Then, following the law of reflection, only backwards, a ray 420 extended from health care provider 412 toward an incidence point of the mirror is determined. Ray 420 may have an angle 402. While diagram 400 only identifies the angle of incidence and reflection for a ray 420 and a ray 422 on a plane for clarity and simplicity, a skilled artisan would recognize that the geometry is three-dimensional, and the drawing describes the angles for these rays on the plane of incidence, which is perpendicular to the reflective surface at point of incidence 424. Based on ray 420, a ray 422 extended from the mirror surface toward an object appearing to the health care provider on the mirror surface is determined. Ray 422 is determined by having an angle 404 equal to angle 402. An incidence point 424 that corresponds to healthcare provider's 412 center of gaze defines the center of the portion of interest out of field of view 208. The center of gaze may be determined by eye tracking methods. A reasonable approximation is to assume that healthcare provider 412 is observing an object at the center of the mirror.

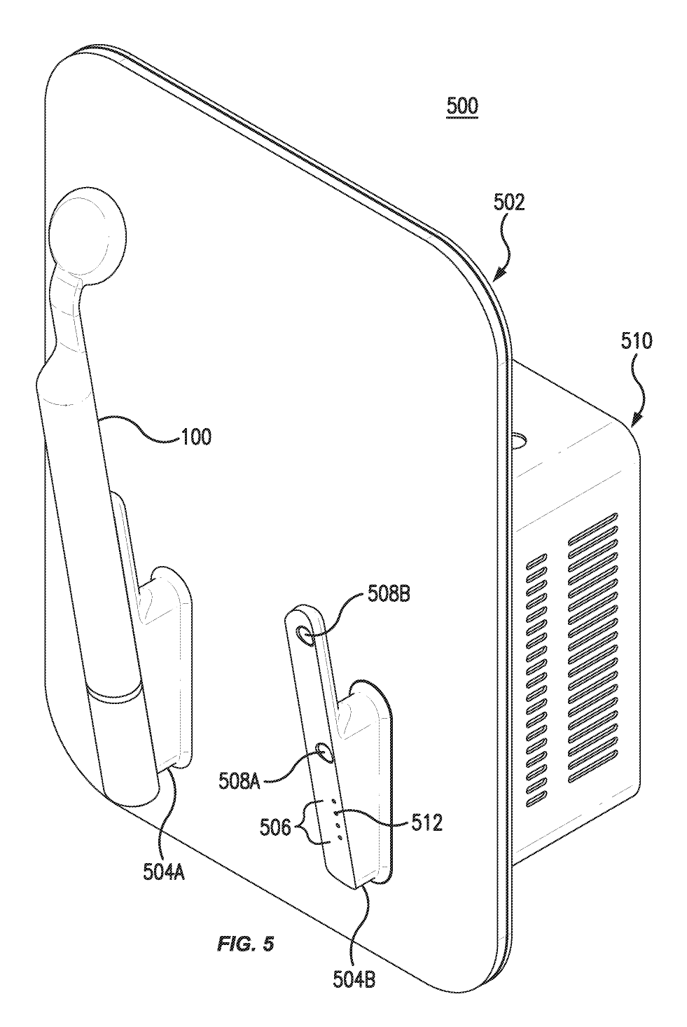

FIG. 5 is a diagram illustrating a base station 500 according to embodiments. In the depicted configuration, base station 500 may be positioned so that smart mirror 100 is substantially perpendicular or substantially parallel to the ground. Base station 500 includes a computer 510 and an attached platform 502. On attached platform 502, base station 500 includes two cradles 504A and 504B, each coupling with a smart mirror device, such as smart mirror device 100.

Each cradle 504 includes a plurality of magnets 508A-B. Magnets 508A-B have opposite polarity with corresponding magnets 150A-B in FIG. 1, such that they attract one another. In this way, magnets 508A-B attach smart mirror device 100 in place on base station 500.

Moreover, each cradle 504 includes a connector 506. Connector 506 may be adapted to couple with connector 152 in FIG. 1, through an electrical connection and, perhaps, a data connection as well. As with connector 152 in FIG. 1, connector 506 includes pins 512 that allow transmission of power, and perhaps data, from base station 500 to smart mirror device 100. Pins 512 may provide a USB connection. For example, connector 506 may include four pins 512, one for voltage (e.g., +5 V), one for ground, and two for data.

Magnets 508A-B are positioned relative to connector 506 in the same manner as magnets 150A-B are positioned relative to connector 152 as shown with respect to smart mirror device 100 in FIG. 1. In this manner, magnets 508A-B hold smart mirror device 100 in such a position as to connect smart mirror device 100's connector 152 with base station 100's connector 506. This allows for a connection to power and to transmit data between base station 500 and smart mirror device 100.

When attached, the data transmitted may include configuration information that configures a secure wireless connection between smart mirror device 100 and base station 500. In this way, unattended wireless connection pairing configuration is enabled. In addition, the data transmitted when attached may include calibration information signaling for smart mirror device 100 to calibrate this gyroscope or accelerometer or to transfer its sensor readings to the base station. While attached to a cradle 504 a smart mirror device 100 is positioned in a stationary orientation with constant attributes such as its angle to the base station plane. Combining the values of these attributes with the readings from sensors may provide a reasonable estimation of the orientation of the mirror, for example, an estimation of a direction towards the ground, which in turn enable the calibration of a gyroscope or accelerometer within smart mirror device 100, or alternatively the calculation of offset values to compensate their future readings, when not attached to the cradle.

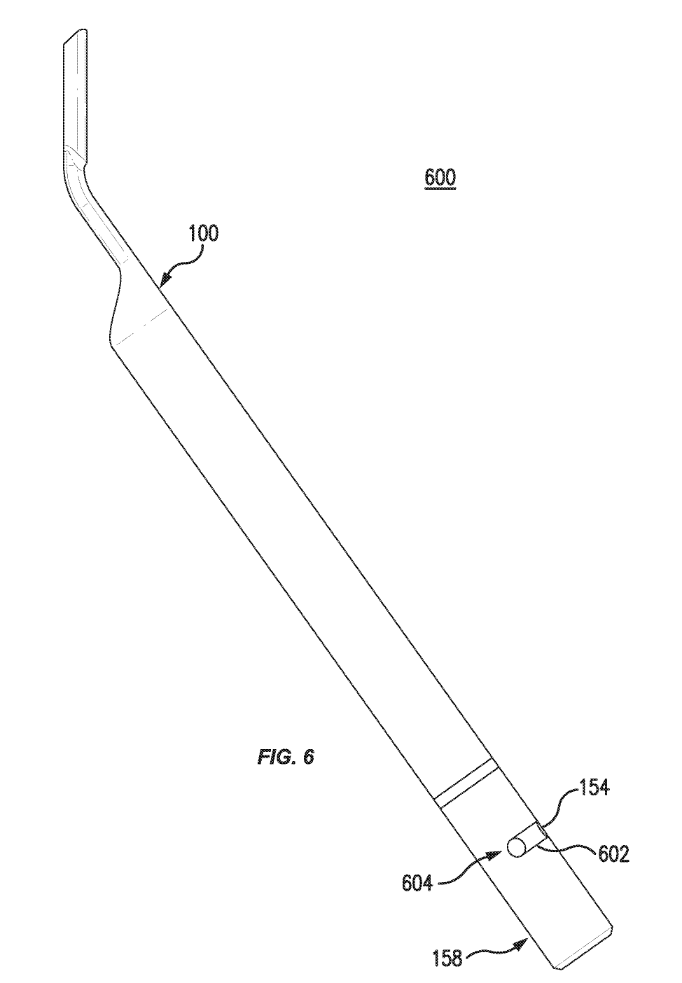

FIG. 6 is a diagram 600 illustrating a cross-section of smart mirror device 100 illustrating how to avoid sound reverberation, according to an embodiment.

As described above, smart mirror device 100 includes hole 154 in end piece 158 that allows sound to enter and reach a microphone, which is illustrated in diagram 600 as microphone 604. Also as mentioned above, end piece 158 and handle 112 (not shown) may be hollow. As sound waves enter hole 154, they may reverberate within the hollow cavity of end piece 158 or handle 112 before reaching microphone 604. This creates an effect as if listening within a tunnel.

To avoid such a tunnel effect, a cylinder 602 is included in smart mirror device 100. Cylinder 602 is hollow and connects the hole and the microphone and configured to avoid, or damp, reverberation of the sound within the hollow cavity of end piece 158 or handle 112. In an embodiment, cylinder 602 may be made of flexible material, such as silicone or rubber. In this way, cylinder 162 directs incoming sound waves from hole 154 to microphone 604.

FIG. 7A is a diagram illustrating a sleeve 700 for an intraoral camera, according to an embodiment. Sleeve 700 may be a disposable, one-time use cover that impermeably covers the mirror surface and a portion of the appendage to enter a patient's mouth. Sleeve 700 includes a head portion 704 and a neck portion 706. Sleeve 700 may be made of silicone, a flexible plastic or rubber material.

Head portion 704 covers the mirror surface. Head portion 704 includes a transparent portion 712 that allows light to and from the mirror surface. The transparent portion covering the reflective area may be a rigid transparent material, such as glass, polycarbonate or acrylic.

Neck portion 706 is impermeably affixed to head portion 704 and includes an opening 710 that allows the disposable cover to slip over the mirror surface and the portion of the appendage to enter the patient's mouth. Neck portion 706 also includes a grip 708 that enables a health care practitioner to hold sleeve 700 and pull it onto smart mirror device 100 as illustrated in FIG. 7B. In preferred embodiments, sleeve 700 should cover the entire appendage, or at least the entire portion of the appendage to enter a patient's mouth.

FIG. 7B is a diagram 750 illustrating smart mirror device 100 with a sleeve 700 attached, according to an embodiment. As illustrated in diagram 750, sleeve 700 covers smart mirror device 100's oral piece 102, of FIG. 1. Sleeve 700 covers smart mirror device 100's oral piece 102, while allowing hand piece 110 to remain exposed.

In this way, by using a single use sleeve 700 to maintain sterility, embodiments avoid the need to autoclave smart mirror device 100. In addition, because sleeve 700 is made of a plastic or rubber material, patients may avoid a metallic feeling present in most dental mirrors.

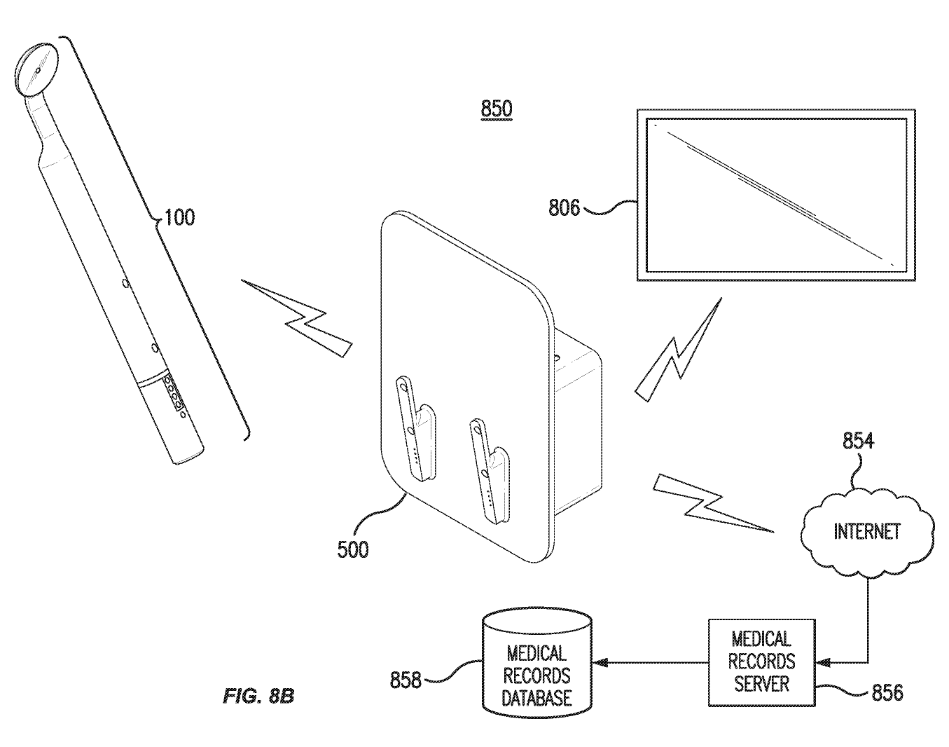

FIG. 8A illustrates a room 800 of a dental office including a system for displaying what is apparent to the healthcare provider in an intraoral mirror. Room 800 shows a healthcare provider 810 utilizing smart mirror device 100 to examine a patient 808. Tablet 806 shows what is visible to the doctor in smart mirror device 100. Also inside room 800 is base station 500. Base station 500 includes cradles 504A-B. Base station 500 allows multiple smart mirrors (not shown) to be used in the same room 800. Base station 500 includes multiple cradles to allow multiple smart mirrors to dock.

Once healthcare provider 810 is no longer using smart mirror device 100, he may place it on one of cradles 504A-B. When smart mirror device 100 is docked with cradles 504A-B, base station 500 may charge smart mirror device 100. Also, when docked with cradles 504A-B, smart mirror device 100 may calibrate its gyroscope and accelerometer.

Base station 500 also provides for communication with smart mirror device 100. In particular, smart mirror device 100 transmits images and other information to base station 500, which transmits the information for display on tablet 806. The communication paths are illustrated in FIG. 8B.

FIG. 8B is an architecture diagram 850 of the system in FIG. 8A. In addition to the components in FIG. 8A, diagram 850 shows a medical records server 856 and a medical records database 858.

Smart mirror device 100 and tablet 806 may be connected using a wireless connection, such as Wi-Fi. In particular, base station 500 may act as a Wi-Fi router and provide network routing and address information to smart mirror device 100 and tablet 806.

Base station 500 is connected to medical records server 856 via one or more networks 854, such as the Internet. Base station 500 may be connected to the Internet either through a wireless or wired LAN in the dental office. Server 856 is a computerized process adapted to run in one or more remote server computers. Server 856 may, for example, be a cloud server. Server 856 is further connected to an archival medical records database 858. Medical records database 858 stores medical record information, including dental status information collected from smart mirror device 100.

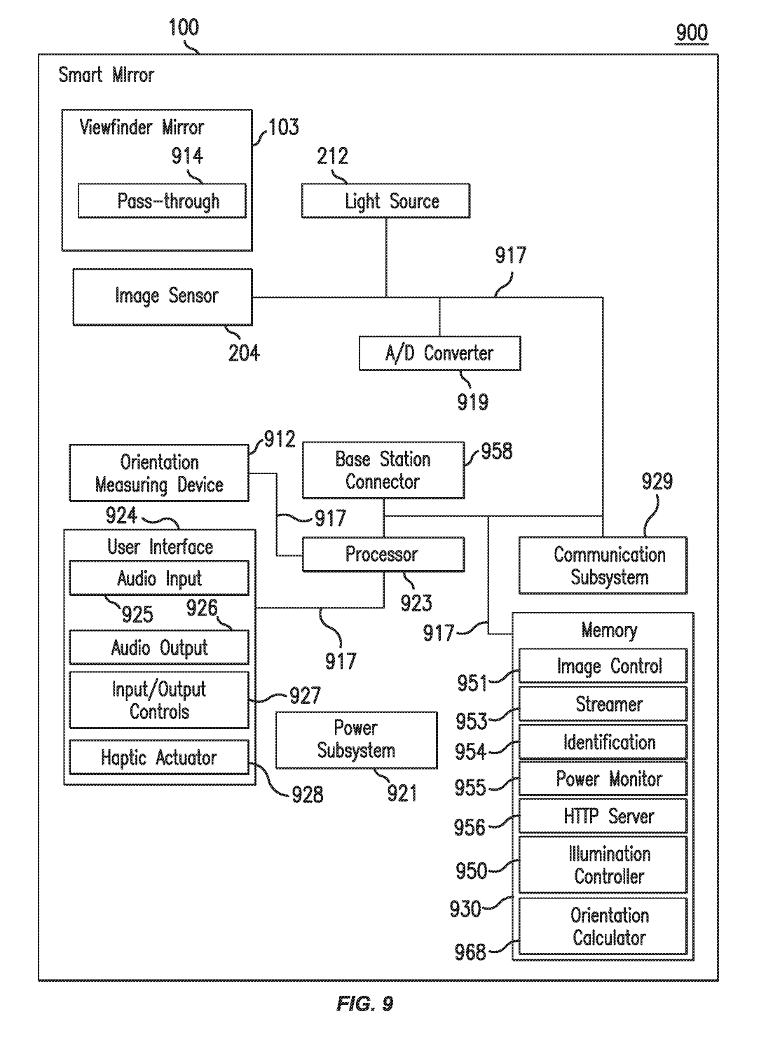

FIG. 9 is a block diagram 900 illustrating a possible configuration of smart mirror device 100.

It may be appreciated for those skilled in the art that a plurality of signal lines or buses 917 may exist, thus different components may be linked by different signal lines or buses 917, and that a signal line or bus 917 depicted in the schematic diagram may represent a plurality of such.

As discussed above for FIG. 1, viewfinder mirror 103 is a mirror, thus having a reflective area. As mentioned above, viewfinder mirror 103 may include electronic components. The reflection from viewfinder mirror 103 provides visual guidance to a user about the objects that may be included in images captured by image sensor 204. As mentioned above, viewfinder mirror 103 may be round. In some embodiments, viewfinder mirror 103 is planar. In some embodiments, viewfinder mirror 103 is curved, concave, convex, or planar. In some embodiments, viewfinder mirror 103 has a spherical shape. In some embodiments, viewfinder mirror 103 has a rectangular shape. It can be appreciated by those skilled in the art that smart mirror device 100 can be embodied with different shapes of viewfinder mirror 103 and/or a plurality of viewfinder mirrors 103 without departing from the spirit of this invention.

Viewfinder mirror 103 includes a pass-through 914. Pass-through 914 allows the pass-through of light or visual information to allow light or visual information to reach image sensor 204 (so that a respective image can be captured) or to allow illumination of objects by light from light source 212. In some embodiments, pass-through 914 is an opening in viewfinder mirror 103. In some embodiments, pass-through 914 is a transparent or semi- or partially-transparent area in viewfinder mirror 103. In some embodiments, pass-through 914 includes an optical lens. In some embodiments, pass-through 914 is a section of the area of viewfinder mirror 103 that becomes transparent or partially transparent when light, possibly of an intensity above some threshold, is present. In some embodiments, pass-through 914 is a section of the area of viewfinder mirror 103 that becomes transparent or partially transparent when electrical current or voltage is present. Pass-through 914 can be located at the center of or at the perimeter, or at other locations of a viewfinder mirror 103.

A plurality of pass-throughs 914 may exist. For example, smart mirror device 100 could include a rounded viewfinder mirror 103 having one pass-through 914 at its center (allowing visual information to reach image sensor 204) and a plurality of pass-throughs 914 equidistant along its perimeter (allowing light from a plurality of light sources 212 to provide illumination).

Image sensor 204 captures still or video digital images. In some embodiments, image sensor 204 is an image sensor, or plurality thereof, that includes a pixel array, such as a charged coupled device (CCD), or a complementary metal-oxide-semiconductor (CMOS) sensor, or the like. An example of an image sensor is the MT9V023 available form ON Semiconductor of Phoenix, Ariz. In some embodiments, image sensor 204 is part of a system-on-chip (SOC) with image sensing capabilities. The SOC may include a memory and/or an image signal processor (ISP) or other components. An example for such a SOC is the OV5640 available from OmniVision Technologies Inc. of Santa Clara, Calif.

As described above, image sensor 204 is located relative to viewfinder mirror 103 so that at least some of the objects reflected in viewfinder mirror 103 are directly visible to image sensor 204 (so that they appear in a captured image). In some embodiments, image sensor 204 is located relative to viewfinder mirror 103 so that at least some of the objects reflected in viewfinder mirror 103 are indirectly visible to image sensor 204. In some embodiments, image sensor 204 is located relative to viewfinder mirror 103 so that at least some of the reflective surface of viewfinder mirror 103 is visible to image sensor 204. In some embodiments, image sensor 204 is located on or adjacent to the reflective area of viewfinder mirror 103. In some embodiments, image sensor 204 is integrated into viewfinder mirror 103. In some embodiments, image sensor 204 is adjacent to pass-through 914. In some embodiments, a lens and/or a light pipe, such as an optical fiber, transmits visual information to image sensor 204. In some embodiments, image sensor 204 is physically separated from the environment (for example an intraoral environment or sterilization environment) by a pass-through 914.

Light source 212 illuminates objects in the proximity of smart mirror device 100. In some embodiments, light source 212 illuminates areas of a person's mouth to improve the image reflected in viewfinder mirror 103 or captured by image sensor 204. In some embodiments, a plurality of light sources 212 are included. In some embodiments, light source 212 emits light. In some embodiments, light source 212 transmits light emitted elsewhere in smart mirror device 100. In some embodiments, the intensity of the light emitted or transmitted by light source 212 can be controlled. In some embodiments, the intensity of illumination by a plurality of light sources 212 is concurrently controlled. In some embodiments, the intensity of each light source 212 of a plurality of light sources 212 is independently controlled. In some embodiments, a plurality of light sources 212 all emit or transmit the same or similar light wavelengths (or colors). In some embodiments, different wavelengths (or colors) may be emitted or transmitted by a plurality of light sources 212. In some embodiments, light source 212 is a led emitting diode (LED). In some embodiments, light source 212 is a light pipe, such as an optical fiber cable or the like. It can be appreciated that other devices can be used as light source 212 to illuminate areas of a mouth without departing from the spirit of the present invention. In some embodiments, light source 212 is a monochromatic light (a laser). In some embodiments, light source 212 transmits light emitted by a laser. In some embodiments, light source 212 is located at a perimeter of viewfinder mirror 103. In some embodiments, light source 212 may be located at a different location of viewfinder mirror 103 and/or elsewhere in oral piece 102 or hand piece 110. In some embodiments, the light emitted and/or transmitted by light source 212 passes through a pass-through 914. In some embodiments, light source 212 is physically separated from the environment (for example an intraoral environment or sterilization environment) by a pass-through 914. In some embodiments, light source 212 is located or directed towards the “back” of viewfinder mirror 103 (further from the non-reflective area) of viewfinder mirror 103, providing ambient illumination as well as illuminating more of the environment space.

Smart mirror 100 further includes an orientation measuring device 912, a user interface 924, a processor 923, a base station connector 958, communication subsystem 929, power subsystem 921, and a memory 930.

Base station connector 958 enables smart mirror 100 to dock with a base station. The docking may occur through a physical connection which holds smart mirror 100 at a predefined orientation. In addition, the docking may occur through a USB or near field communication connection or the like. When docking with the base station, smart mirror 100 may receive electrical power through base station connector 958, which may be used to charge power subsystem 921. In addition, smart mirror 100 may receive control and signaling information through base station connector 958. For example, base station connector 958 may receive information needed to configure a wireless communication connection between smart mirror 100 and the base station. Base station connector 958 may provide the wireless configuration information (such as a service set identifier and password) to communication subsystem 929, as is discussed below. And, when docked to a base station, base station connector 958 may signal orientation measuring device 912 or software in memory 930 to calibrate.

Power subsystem 921 stores power for smart mirror device 100 and provides power to the other components of smart mirror device 100. Power subsystem 921 may include batteries, such as AAAA batteries, or a capacitor.

Orientation measuring device 912 measures an orientation (including x,y,z, position and yaw, pitch, roll direction) of viewfinder mirror 103 or generates data that enables to calculate an orientation of viewfinder mirror 103. In some embodiments, orientation measuring device 912 is an accelerometer. An example of an accelerometer is MMA8453Q available from NXP Semiconductors N.V. of Eindhoven, Netherlands. In some embodiments, orientation measuring device 912 is a gyroscope. An example of a gyroscope is FXAS21002C also available from NXP Semiconductors N.V. In some embodiments, orientation measuring device 912 is a magnetometer. An example of a magnetometer is MAG3110 also available from NXP Semiconductors N.V. Sometimes two or more of such sensors are combined into one, for example the imu (inertial measurement unit) LSM9DS1 available from ST Microelectronics.

User interface 924 includes an audio input 925, audio output 926, and input/output controls 927. Audio input 925 captures audial information. In some embodiments, audio input 925 includes a microphone. In some embodiments, audio input 925 captures human voice, for example, to enable a healthcare provider to dictate observations for a patient's medical record. Smart mirror 100 includes an audio output 926, which emits sounds. In some embodiments, audio output 926 includes one or more speakers. In some embodiments, audio output 926 includes headphone jacks and/or headphones.

Input/output controls 927 can include buttons, lights, knobs, capacitive sensors, or the like for a user to control and/or receive feedback relating to processes in smart mirror device 100, for example, to initiate audio recording or image capturing, or set an intensity of illumination.

Haptic actuator 928 provides a light vibration to signal the user of various conditions. In an embodiment, haptic actuator 928 may engage to provide a vibration to indicate that the smart mirror 100 is about to shutdown or turn to a power save mode. Haptic actuator 928 may, for example, be a linear resonant actuator vibration motor or an eccentric rotating mass (ERM) vibration motor.

Communication subsystem 929 allows smart mirror 100 to connect to one or more remote computational devices, including, for example, to a base station, or to a general purpose computational device such as personal computer, a smart phone, a tablet or similar, or a specialized computational device such as to another smart mirror device or remote speakers or the like. In some embodiments, communication subsystem 929 is adapted to connect to a wireless network, including, but not limited to, WiFi and/or Bluetooth. In some embodiments, communication subsystem 929 is adapted to attach to a wired network, including, but not limited to, Ethernet, USB or thunderbolt.

Memory 930 may include random access memory (RAM) and may also include nonvolatile memory, such as read only memory (ROM) and/or flash memory. Memory 930 may be embodied as an independent memory component, and may also be embedded in another component, such as processor 923 and/or image sensor 204, or may be embodied as a combination of independent as well as embedded, and/or a plurality of memory components is present. Memory 930 is adapted to include software modules (a module is a set of instructions). In particular, memory 930 includes a streamer module 953, identification module 954, power monitor module 955, HTTP server module 956, illumination controller module 950, image control module 951, and orientation calculator module 968.

Processor 923 is adapted to run instructions stored in memory 930. Processor 923 may be a micro-controller unit (MCU), a digital signal processor (DSP) and/or an Image/Video Processing unit or the like components that run instructions. An example of an MCU is MSP432P401x available from Texas Instruments Inc. of Dallas, Tex. An example of a DSP is C5000 available from Texas Instruments Inc. of Dallas, Tex. An example of an image/video processor is OMAP3525 available from Texas Instruments Inc. of Dallas, Tex. One or more of processor 923 may be present. Processor 923 may be an independent component, it may also be embedded in another component, such as in image sensor 204, or any combination thereof.

In embodiments, smart mirror 100 includes an analog to digital converter 919 (A/D). The analog to digital converter 919 may convert imagery information output from the image sensor from an analog representation to a digital representation. The smallest, best available image sensors may output images using an analog signal. The analog signal may be a wave ranging from 0 to 3.3 V. A/D converter 919 samples the analog output from image sensor 204 and converts the sampled readings into digital values. The digital values together comprise a digital representation of images captured by image sensor 204 and can be used and interpreted by other components such as processor 923.

Illumination controller module 950 controls the operation of light source 212. In some embodiments, illumination controller module 950 sets the intensity of illumination of light source 212. In some embodiments, illumination controller module 950 receives a user request to increase or reduce illumination. In some embodiments, illumination controller module 950 receives a user request to turn on or off some or all of light source 212. In some embodiments, illumination controller module 950 receives requests from other software modules to increase and/or decrease illumination of one or more of light source 212. In some embodiments, user input as well as said requests are used to determine an intensity of illumination.

Orientation calculator module 968 reads data from orientation measuring device 912. Orientation calculator module 968 may for example integrate data from a gyroscope and accelerometer, and possibly a magnetometer to determine a location (in, for example, x,y,z coordinates) and a direction (for example, yaw, pitch, and roll). Because orientation calculator module 968 uses integration to determine the location and direction of smart mirror device 100, errors from the gyroscope and the accelerometer can accumulate over time. However, as described above, base station connector 958 may dock with the base station in such a way to position smart mirror 100 at a known angle. When base station connector 958 is docked with the base station, base station connector 958 may signal orientation calculator module 968 to calibrate. To calibrate, orientation calculator module 968 may set the x, y, z, and yaw, pitch, and roll values to fixed values, such as the value zero. Thus, when smart mirror device 100 is moved around, the coordinate and direction values orientation calculator module 968 determines may be relative to the coordinate and direction values set at the base station. Also, to calibrate, the sensory readings may be transferred through base station connector 958, to let the base station their values while docked, so that future readings can be compensated for errors.

Image control module 951 controls the capture of images and video, and affects the output image quality. In some embodiments, image control module 951 controls the intensity of illumination, for example, by requests to illumination module 950, for example to improve the illumination conditions for a better image capture quality. In some embodiments, image control module 951 processes a set of time-successive images to create a single output image which has an improved visual quality, for example, but not limited to by selecting one image out of the set, or by combining portions of images, each portion from an image in the set. In some embodiments, values indicating the acceleration of image sensor 204 when an image was captured are used to improve the quality of an output image, for example, but not limited to, selecting images with least acceleration or interpolating among portions of two or more images of different acceleration. In some embodiments, image control module 951 controls the aperture and/or focal point of a lens. In some embodiments, image control module 951 triggers the capture of a sequence of images each with a different illumination. In some embodiments, image control module 951 triggers the capture of a sequence of images each with a possibly different group of one or more of light sources 212 set to illuminate, while the other one or more of light source 212 set to not illuminate. In some embodiments, image control module 951 rotates an image, for example based on a rotation value generated by orientation calculator module 968.

Identification module 954 identifies smart mirror device 100 to a remote computational device. In some embodiments, identification module 954 implements an authentication handshake protocol in which the identification occurs over a network session. In some embodiments, identification module 954 couples an identification to data prior to the data being transferred to a remote computational device. The identification may include a globally unique ID for smart mirror 100. It may also be timestamped and digitally signed.

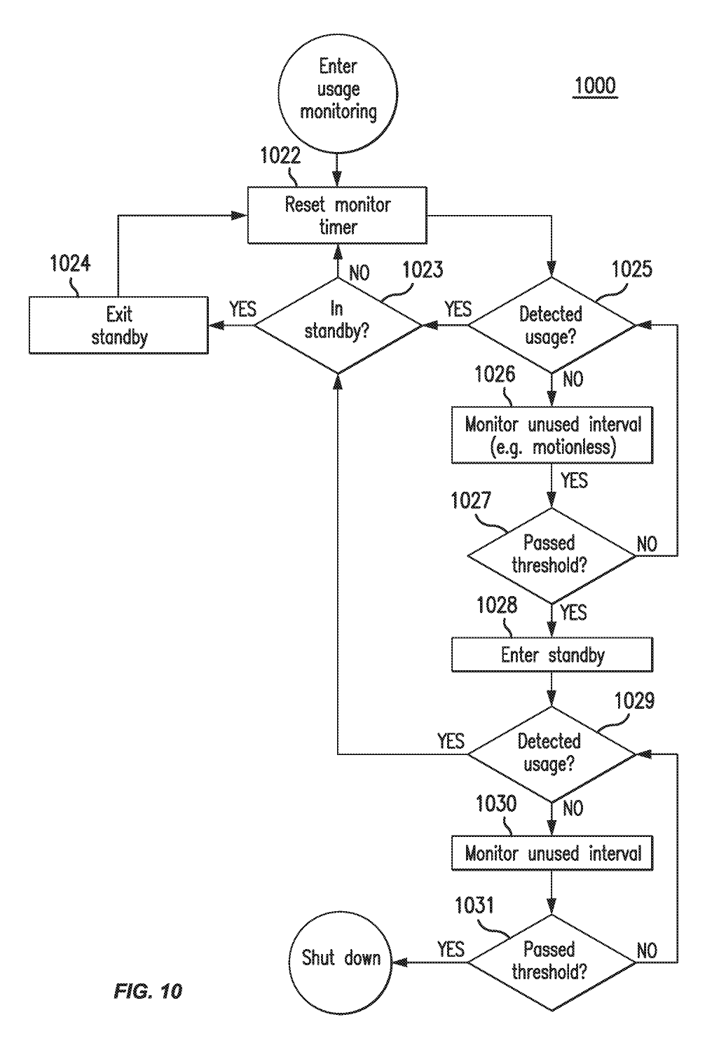

Power monitor module 955 monitors the amount of energy available by the power subsystem, and the power usage of smart mirror device 100. In some embodiments, power monitor module 955 receives a motion indication generated by orientation measuring device 912, for example, but not limited to, an acceleration indication. In some embodiments, power monitor module 955 sets smart mirror device 100 into a standby mode when smart mirror device 100 is not being used for a time interval larger than some threshold. In another embodiment, power monitor module may operate as described below with respect to FIG. 10. To set a standby mode, in which smart mirror device 100 consumes a reduced amount of power, power monitor module 955 may reduce or completely shut down the power supply to some of smart mirror device 100's components and/or alter or completely pause some of smart mirror device 100's software processes, or the like. In any of these situations, power monitor module 955 may signal haptic actuator 928 to provide a vibration signaling to the user of the state change. In some embodiments, power monitor module 955 exits a standby mode, for example, by resuming power to some of smart mirror device 100's components or resuming execution of some of smart mirror device 100's processes, when an indication of usage of smart mirror device 100 is present. In some embodiments, power monitor module 955 enters or exits a standby mode based on other parameters or indications, the invention is not so limited. In some embodiments, power monitor module 955 performs a shutdown, shutting power to more (when compared to standby mode) or even all of smart mirror device 100's components, when an indication of not being used is present for a time interval larger than some threshold, or based on other parameters or indications.

Streamer module 953 prepares and/or streams data to a remote computational device. The data can include video collected from image sensor 204, smart mirror device 100's orientation and location and other information collected from or generated by orientation calculator module 968, audio input collected from audio input 925, any data collected from input/output controls 927, power related data collected from power monitor 955 and the specification of how light source 212 is illuminated from illumination controller module 950. Streamer module 953 may associate data collected from these various sources with each other. To associate data collected from different sources, streamer module 953 may attach a timestamp. For example, each frame in video image sensor 204 may include a timestamp indicating the time it was collected. Similarly, the orientation, audio, power, and input control information may have a timestamp indicating when that information was collected, and the illumination information may have a timestamp indicating when light source 212 was illuminated in the manner specified.

In some embodiments, streamer module 953 formats images, video, audio and other data in a format for streaming to an application executing on a remote computational device via communication subsystem 929. In some embodiments, streamer module 953 formats images, video, audio and other data in a format suitable for streaming to an Internet browser, for example, but not limited to, HTTP streaming, HTML, HTML5, RTSP, WebRTC, or other standard formats such as RTP. In some embodiments, streamer module 953 formats images, video, audio and other data with compression formats and/or format containers such as, but not limited to, JPG, JPG 2000, MPEG-4, H.264, H.265, AAC, PCM, G.711, G.726, and the like. In some embodiments a proprietary format is used, the invention is not so limited.

In an embodiment, processor 923 is configured to analyze signals received from base station connector 958 to determine when the dental mirror no longer is docked to the base station. For example, processor 923 may determine when base station connector 958 is no longer receiving power. When processor 923 determines that the dental mirror no longer is docked to the base station, processor 923 is configured to instruct streamer module 953, and in turn communication subsystem 929, to stream video captured image sensor for display on a user interface, such as a user interface on tablet 806.

In addition to streamer module 953, smart mirror 100 may transmit data using a HTTP server 956. In some embodiments, HTTP server 956 responds to HTTP requests originating in a remote computational device.

FIG. 10 illustrates a method 1000 for monitoring usage of the intraoral mirror to conserve power.

At step 1022, an “unused interval” timer, indicating a length of a time interval in which the smart mirror device is not being used, is reset. At step 1025, usage detection is validated. In some embodiments, usage detection includes motion detection. In some embodiments, motion detection is performed by processing measurements of acceleration of the smart mirror in one or more axes. In some embodiments, motion detection is performed by processing measurement of changes in angles of the smart mirror relative to one or more axes. In some embodiments, motion detection is performed by processing a set of temporally successive images captured by the smart mirror, for example, identifying changes among the images. In some embodiments, motion detection is performed by sensing detachment from the base station. In may be appreciated that various ways to detect motion of the smart mirror exist, the invention is not so limited. In some embodiments, usage detection includes analyzing the orientation of the smart mirror, for example checking if it lays horizontally (if so it might be left on table or a tray). In some embodiments, usage detection includes processing images captured by the smart mirror, for example to assert that viewfinder mirror is in an intraoral environment. In some embodiments, usage detection includes monitoring the wireless communication. In some embodiments, a plurality of such condition validations are used to determine whether the smart mirror is in use.

At step 1026, usage has not been detected, and an unused interval is determined. At step 1027, said interval is compared to a threshold. At step 1028, said interval surpasses a threshold, and smart mirror is set to a standby state in which its energy consumption is reduced, for example, by reducing or cutting off the energy supply to one or more of its components and/or suppressing or altering one or more of its software processes.

At step 1029, usage detection is further validated. At step 1030, usage has not been detected, and an unused interval is calculated, for a period in which the smart mirror is in standby mode. At step 1031, said interval is compared to a threshold. If the interval surpasses the threshold, the smart mirror is shut down to further reduce its energy consumption, for example, by reducing or cutting off the energy supply to one or more or all of its components and/or suppressing or altering one or more or all of its software processes. At step 1023, usage has been detected and power mode is standby is checked.

At step 1024, smart mirror exits standby mode including, but not limited to, resuming power to one or more of its components and/or resuming one or more of its software processes.

The databases disclosed herein may be any stored type of structured memory, including a persistent memory. In examples, this database may be implemented as a relational database or file system.

Each of the processors and modules in FIG. 9 may be implemented in hardware, software, firmware, or any combination thereof implemented on a computing device. A computer or computing device can include, but is not limited to, a device having a processor and memory, including a non-transitory memory, for executing and storing instructions. The memory may tangibly embody the data and program instructions. Software may include one or more applications and an operating system. Hardware can include, but is not limited to, a processor, a memory, and a graphical user interface display. The computing device may also have multiple processors and multiple shared or separate memory components. For example, the computing device may be a part of or the entirety of a clustered or distributed computing environment or server farm.

Identifiers, such as “(a),” “(b),” “(i),” “(ii),” etc., are sometimes used for different elements or steps. These identifiers are used for clarity and do not necessarily designate an order for the elements or steps.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

What is claimed is:

1. A dental mirror, comprising:
an image sensor;
a plurality of light sources;
a mirror surface, wherein the mirror surface comprises:
a transparent planar material covering the image sensor and the plurality of light sources, and
a first reflective coating portion on the transparent planar material, the first reflective coating portion positioned to reflect light into a health care provider's field of view, wherein the first reflective coating portion includes an opening positioned to allow the image sensor to capture an image of what is reflected into the health care provider's field of view,
wherein the plurality of light sources are positioned around a perimeter of the mirror surface close enough to one another to emit a continuous ring of light;
an appendage affixed to the mirror surface to extend the mirror surface into a patient's mouth; and
a handle affixed to the appendage.

2. The dental mirror of claim 1, wherein the plurality of light sources are spaced no more than 18 degrees from one another.

3. The dental mirror of claim 1, wherein the transparent planar material is synthetic sapphire.

4. The dental mirror of claim 1, wherein the first reflective coating portion is aluminum.

5. The dental mirror of claim 1, further comprising a second reflective coating portion on the transparent planar material positioned around the perimeter of the mirror surface to allow the plurality of light sources to emit light through the second reflective coating portion to illuminate what is reflected into the health care provider's field of view

6. The dental mirror of claim 5, wherein the second reflective coating portion is aluminum that: (i) reflects light into the health care provider's field of view; and (ii) is applied non-continuously with discontinuities allowing the passage of light emitted from the plurality of light sources when the plurality of light sources is illuminated.

7. The dental mirror of claim 6, wherein the second reflective coating portion is less than 1.5 mm wide.

8. The dental mirror of claim 1, further comprising an analog-to-digital converter that converts analog signals from the image sensor to digital signals for use by the processor.

9. The dental mirror of claim 1, wherein the first reflective coating portion is applied on an interior side of the transparent planar material.

10. The dental mirror of claim 1, wherein the handle has an elongated oval shape configured to allow the health care provider to grip the dental mirror.

11. The dental mirror of claim 1, wherein each light source of the plurality of light sources is at least one light emitting diode.

12. The dental mirror of claim 1, further comprising a lens positioned to refract light through the opening to the image sensor, wherein the image sensor, lens, and opening are positioned substantially at the center of the mirror surface.

13. The dental mirror of claim 13, wherein the mirror surface is round and attached to the appendage at an angle to the handle.

14. The dental mirror of claim 1, further comprising a disposable cover that impermeably covers the mirror surface and a portion of the appendage to enter the patient's mouth.

15. The dental mirror of claim 15, wherein the disposable cover comprises:
a head portion that covers the mirror surface, the head portion including a transparent portion that allows light to and from the mirror surface; and
a neck portion impermeably affixed to the mirror surface with an opening that allows the disposable cover to slip over the mirror surface and the portion of the appendage to enter the patient's mouth.

16. The dental mirror of claim 1, further comprising:
a wireless interface;
a processor; and
a power supply configured to power the wireless interface, the processor, the plurality of light sources and the image sensor.

17. The dental mirror of claim 17, further comprising:
an interface configured to, when the dental mirror is docked to a base station, receive electrical power from the base station and to store the electrical power in the power supply.

18. The dental mirror of claim 17, wherein the processor is configured to: (i) analyze signals received from the interface to determine when the dental mirror no longer is docked to the base station, and (ii) when the processor determines that the dental mirror no longer is docked to the base station, instructs the wireless interface to stream video captured image sensor for display on a user interface.

19. The dental mirror of claim 17, further comprising:
a magnet to attach the dental mirror to the base station when the dental mirror is docked.

20. The dental mirror of claim 17, wherein the processor is configured to determine when the dental mirror is about to shut down, further comprising:
an actuator, coupled to the processor, that is configured to provide haptic feedback when the dental mirror is about to shut down.

21. The dental mirror of claim 17, further comprising:
a microphone, coupled to the processor; and
a hollow cavity within the handle that contains the microphone, processor, power supply, and wireless interface, wherein the hollow cavity has a hole to allow sound to reach the microphone.

22. The dental mirror of claim 21, further comprising:
a cylinder connecting the hole and the microphone, the cylinder configured to avoid reverberation of the sound within the hollow cavity.

23. The dental mirror of claim 1, wherein the mirror surface is less than 1 mm in thickness.

24. The dental mirror of claim 1, wherein the mirror surface is circular and has a diameter between 18 mm and 24 mm.

25. The dental mirror of claim 1, wherein the opening is substantially circular and located substantially at the center of the mirror surface.

26. The dental mirror of claim 25, wherein the opening has a diameter between 0.8 mm and 1.3 mm.

27. A disposable cover for a dental mirror, comprising:
a head portion configured to cover a mirror surface that includes (i) a transparent planar material covering an image sensor affixed to the dental mirror and a plurality of light sources affixed to the dental mirror and positioned around a perimeter of the mirror surface close enough to one another to emit a continuous ring of light, and (ii) a reflective coating portion on the transparent planar material, the reflective coating portion positioned to reflect light into a health care provider's field of view, wherein the reflective coating portion includes an opening positioned to allow the image sensor to capture an image of what is reflected into the health care provider's field of view; and
a neck portion impermeably affixed to the mirror surface with an opening that allows the disposable cover to slip over the mirror surface and a portion of an appendage to enter a patient's mouth.

28. A base station, comprising:
a computing device; and
a cradle, coupled to the computing device, configured to interface with a dental mirror that includes an image sensor, a plurality of light sources positioned around a perimeter of a mirror surface close enough to one another to emit a continuous ring of light, and the mirror surface, wherein the mirror surface comprises (i) a transparent planar material covering the image sensor and the plurality of light sources, (ii) a reflective coating portion on the transparent planar material, the reflective coating portion positioned to reflect light into a health care provider's field of view, wherein the reflective coating portion includes an opening positioned to allow the image sensor to capture an image of what is reflected into the health care provider's field of view.

29. The base station of claim 28, wherein the cradle comprises:
a magnet positioned on the cradle to attach to an opposing magnet on the dental mirror.

30. The base station of claim 28, wherein the cradle comprises:
a connector to electrically couple with the dental mirror.
